(19) United States
(12) Patent Application Publication
(10) Pub. No.: US 2004/0229285 A1
(43) Pub. Date: Nov. 18, 2004

(54) Serotonin and catecholamine system segment optimization technology

(76) Inventor: Martin C. Hinz, Duluth, MN (US)

Correspondence Address: JOEL D. SKINNER, JR., SKINNER AND ASSOCIATES, 212 COMMERCIAL ST., HUDSON, WI, 54016, US

(21) Appl. No.: 10/785,158

(22) Filed: Feb. 23, 2004

Related U.S. Application Data
(60) Provisional application No. 60/449,229, filed on Feb. 21, 2003.

(51) Int. Cl. G01N 33/53

(52) U.S. Cl. 435/7.1; 514/419; 514/565

(57) ABSTRACT

A method of treating neurotransmitter dysfunction in a patient by administering amino acid precursors in conjunction with laboratory assay of the neurotransmitters. The method includes the step of administering an amino acid precursor of a catecholamine in a balanced and effective therapeutic range. The catecholamine precursor is preferably L-dopa, but may alternatively be tyrosine, D,L-Phenylalanine or an active isomer thereof, and N-acetyl-L-tyrosine or other amino acid precursor of L-dopa. An amino acid precursor of serotonin in an effective therapeutic range, is also administered. The serotonin precursor is preferably 5-HTP, but may alternatively be tryptophan. At least one cofactor is also preferably administered. Cofactor options include Vitamin B6, Vitamin C, Calcium, Folate, and Cysteine. A method of periodic administration and patient checking is also disclosed.

### CROSS-REFERENCE TO RELATED APPLICATIONS, IF ANY

This application claims the benefit under 35 U.S.C. § 119(e) of co-pending U.S. Provisional Patent Application Serial No 60/449,229 filed Feb. 21, 2003, which is incorporated by reference.

### 37 C.F.R. § 1.71(e) AUTHORIZATION

A portion of the disclosure of this patent document contains material which is subject to copyright protection. The copyright owner has no objection to the facsimile reproduction by anyone of the patent document or the patent disclosure, as it appears in the US Patent and Trademark Office patent file or records, but otherwise reserves all copyright rights whatsoever.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### BACKGROUND

1. Field

The present invention relates, generally, to biomedical technology. More particularly, the invention relates to a technology for optimizing the serotonin and catecholamine systems by administration of amino acid precursors in conjunction with laboratory assay of the neurotransmitters of the catecholamine and serotonin systems. Most particularly, the invention relates to safe, effective compositions, methods and therapies for balancing, treating and optimizing the serotonin and catecholamine neurotransmitter systems in humans as guided by laboratory testing of neurotransmitters. The compositions, methods and techniques of the invention have broad applicability with respect to neurotransmitter dysfunction, including disease. The compositions, methods, and techniques may also be useful in other fields.

2. Background Information

With administration of amino acid precursors of the serotonin and/or catecholamine system there is an increase in neurotransmitter levels of those systems in the system as a whole. Prior to this work laboratory measurement of neurotransmitters of the catecholamine and serotonin systems (hereafter referred to as “The System”) in correlation with amino acid response for clinical applications had not been calibrated for use. The present invention provides a methodology for performing meaningful neurotransmitter laboratory assay in support of amino acid therapy in the treatment of neurotransmitter dysfunction of the serotonin and catecholamine system. This invention is a preferred step in any process where manipulations of the serotonin and catecholamine systems are of consideration.

Neurotransmitter dysfunction associated with the catecholamine and/or serotonin system may include, but is not limited to, depression, anxiety, panic attacks, migraine headache, obesity, bulimia, anorexia, premenstrual syndrome, menopause, insomnia, hyperactivity, attention deficit disorder, impulsivity, obsessionality, aggression, inappropriate anger, psychotic illness, obsessive compulsive disorder, fibromyalgia, chronic fatigue syndrome, chronic pain states, adrenal fatigue, attention deficit hyperactivity disorder, Parkinsonism, and states of decreased cognitive function such as dementia and Alzheimer's disease.

Applicant has disclosed therapies for treatment of obesity and for serotonin and catecholamine system segment optimization technology in the following US patents and published US patent applications: U.S. Pat. No. 6,660,777 issued Dec. 9, 2003; U.S. Pat. No. 6,548,551, issued Apr. 15, 2003; U.S. Pat. No. 6,403,657, issued Jun. 11, 2002; U.S. Pat. No. 6,384,088, issued May 7, 2002; US Patent Application Publication No. US 2003/0181509 A1, published Sep. 25, 2003; US Patent Application Publication No. US 2002/0094969 A1, published Jul. 18, 2002; US Patent Application Publication No. US 2002/0072537 A1, published Jun. 13, 2002; US Patent Application Publication No. US 2002/0065311 A1, published May 30, 2002; US Patent Application Publication No. US 2002/0040054 A1, published Apr. 4, 2002; and US Patent Application Publication No. US 2002/0025972, published Feb. 28, 2002.

A need is believed to exist for the present invention. All US patents and patent applications, and all other published documents mentioned anywhere in this application are hereby incorporated by reference in their entirety.

### BRIEF SUMMARY

An understanding the following basic chemical properties of amino acids is helpful to understand laboratory assay of the response to amino acids by the system.

Amino acids of interest include 3-Hydroxy-L-,t,tyrosine (hereafter referred to as “L-dopa”) and 5-hydroxytryptophan (hereafter referred to as “5-HTP”). L-dopa is an amino acid precursor of the catecholamine system (dopamine, norepinephrine, and epinephrine) and 5-HTP is an amino acid precursor of serotonin. Both share unique chemical properties in the human body and other higher forms of life, including:

1. Both can be absorbed into the system significant amounts after oral ingestion.

2. Being water soluble both cross the blood brain barrier freely.

3. L-dopa is freely converted to dopamine and 5-HTP is freely converted to serotonin when exposed to the enzyme which catalyzes the synthesis of dopamine or serotonin in conjunction with required cofactors.

4. Neither is subject to biochemical regulation in the synthesis of dopamine and serotonin giving the unique ability to establish theoretical neurotransmitter levels of dopamine and serotonin in unlimited amounts.

Other amino acid precursors include tryptophan of the serotonin system and include but are not limited to tyrosine, N-acetyl-1-tyrosine, and phenylalanine of the catecholamine systems. These amino acids share the same chemical properties as L-dopa and 5-HTP with the exception that they are subject to biochemical feedback regulation meaning that only a limited amount of dopamine and serotonin can be produced by the system with their administration. Production of tryptophan is regulated via the, “serotonin/tryptophan hydroxylase feedback loop” and production of L-dopa from tryosine is regulated via the, “norepinephrine/tyrosine hydroxylase feed back loop”.

Laboratory assay of neurotransmitters of the serotonin and catecholamine systems can be carried out by assay of serum, saliva, urine, or any other method which accurately reflects the neurotransmitter levels of the system. Based on the following discussion, the preferred method is urinary assay.

With regards to neurotransmitter assay of serum a major limitation is the collection of a sample. It is a fact that neurotransmitter levels fluctuate greatly from minute to minute and the mere act of inducing a needle into a subject causes an instantaneous spike in the neurotransmitter levels of the system which makes it impossible to obtain accurate neurotransmitter level readings that are reflective of levels just prior to insertion of the needle. Methods to compensate for this limitation are cumbersome to the point of not being useful in routine evaluation of subjects. For example, to obtain a true baseline neurotransmitter reading from serum the subject should have a central venous catheter inserted and be allowed to lie quietly in a darkened room for 30 minutes at which point a serum sample can be drawn as long as the subject is not disturbed. A second method of obtaining a serum sample that is meaningful in the assay of serum neurotransmitters levels is to place an indwelling catheter in the subject and allow the subject several days to get used to the catheter at which point serum can be obtained. These two methods are not intended to be an exhaustive discussion on the methodology for obtaining valid serum specimens but are intended to illustrate the considerations that need to be made if serum assay of neurotransmitters in support of amino acid administration is opted for.

Salivary assay may be an option but again saliva is subject to minute to minute variability of the system as a whole. A method to compensate for the fluctuations in the system is to obtain several (four to six) saliva samples during an approximate time period of 30 minutes then to average the results of the samples. The drawback of this method is that the final reported assay is a coarse approximation at best and the cost is higher since four to six independent tests need to be run, plus it requires specimen collection over a 30 minute period of time without interruption.

The method opted for as the method of choice in assay of neurotransmitter levels is urinary neurotransmitter testing. This assay is not a completely straight forward assay either and must be preformed with adherence to the following considerations. In reporting urinary assay results consideration must be made to compensate for dilution of the urine (specific gravity variance). Simply assaying the neurotransmitters in a given urine sample will not give results of desired meaning due to variance in specific gravity from sample to sample. One method to compensate for variance in specific gravity is to report the results as a “neurotransmitter to creatinine ratio”. The preferred method is reporting results as micrograms of neurotransmitter per gram of creatinine in the urine. In utilizing urinary laboratory assay of neurotransmitters the problem of minute to minute spikes in the neurotransmitter levels is overcome and the results reported are an average of the neurotransmitters levels in the urine since the bladder was last emptied (generally 2 to 3 hours earlier). Other considerations of urinary neurotransmitter assay include but are not limited to the urine should not be collected first thing in the morning unless you are assaying neurotransmitter levels during the night. Contrary to the usual method for collection urine for neurotransmitter assay where a pathologic diagnosis of pheochromocytoma, serotonin secreting tumor, and the like is being made the urine used in assay of neurotransmitters in support of amino acid therapy of The System should be collected late in the day (preferably 5 to 6 hours before bed time) when the neurotransmitter levels are at their lowest. In the case where pathologic diagnosis is being made or in lab testing to assist in establishing neurotransmitter levels in the optimal range throughout the day, or to gauge situations of neurotransmitter overload and toxicity it is desirable to collect urine in the AM when neurotransmitter levels are at their highest so as to demonstrate peak levels. Urinary assay of neurotransmitters in support of amino acid therapy of The System should be collected at or near the low point 5 or 6 hours before bed time to insure that a neurotransmitter assay is obtained in an effort to insure that neurotransmitter levels do not drop below levels needed to keep the system free of disease symptoms (a therapeutic range).

The term “hyperexcretion” is used for circumstance where the urinary neurotransmitter assay in a subject not under treatment with amino acids is higher than the systemic neurotransmitter assay as verified by salivary or serum assay of neurotransmitters. It has been demonstrated that 23.7% of human subjects tested late in the day (5 to 6 hours before bed time) are hyperexcreting epinephrine. Hyperexcretion appears to be due to inappropriate excretion of neurotransmitters by the kidneys. Hyperexcretion of neurotransmitters also occurs with serotonin, norepinephrine, and dopamine. Hyperexcretion is a consideration in base line testing of subjects prior to initiation of amino acid treatment and the impact of hyperexcretion must be considered during interpretation of these tests.

Once a subject with hyperexcretion is under treatment with amino acids (generally in a matter of a few days, 3 to 5 days for dopamine and serotonin and can take up to 6 to 8 months for epinephrine and norepinephrine) the hyperexcretion problem starts to resolve and a more positive correlation between the urinary neurotransmitter assay and the systemic neurotransmitters assay is seen leading to more meaningful results in establishing “optimal and therapeutic ranges” which are discussed further below.

The primary application of laboratory assay of neurotransmitters of The System is to assist in establishing therapeutic levels of neurotransmitters. Referring to FIG. 1 the following discussion is put forth. We use the 5-HTP component of the balanced administration of amino acids for this discussion. Similar considerations exist for L-dopa and dopamine and other amino acid precursors of The System. The horizontal axis of FIG. 1 represents the daily milligram dosing of 5-HTP in 100s of milligrams. The vertical access of FIG. 1 represents urinary serotonin levels as reported in micrograms per gram of creatinine. In a subject who is treated with balanced amino acids and has laboratory assay of neurotransmitter performed frequently (with every 100 milligram increase in 5-HTP) the “dose-response curve” of FIG. 1 is typical. Urinary neurotransmitter levels of FIG. 1 do not change and are flat until the “inflection point” is arrived at which in this case is at the daily dosing of approximately 800 milligrams per day of 5-HTP. Once the inflection point is arrived at small increases in the amino acid precursors dosing levels lead to large increases in the urinary neurotransmitter levels. In recognizing this rapid upward inflection of the urinary neurotransmitter levels we are able to define a “therapeutic range” in the treatment of neurotransmitter dysfunction disease symptoms. It is noted that the amino acid dosing of precursors needs of both the catecholamine and serotonin systems vary widely in a group of subjects with regards to dosing at which the inflection point occurs. The exact urinary neurotransmitter levels of the optimal and therapeutic range may vary depending on the methodology of the laboratory doing the assay but in general the lower limits of the range need to be high enough to insure that symptoms of neurotransmitter dysfunction are under control and to top end of the therapeutic range needs to be set at such a level as to insure that the subject is not being over loaded with neurotransmitter during treatment leading to undesirable outcomes. For the purposes of the illustration in FIG. 1 the therapeutic range for serotonin is set at 800 micrograms of neurotransmitter per gram of creatinine. Other ranges exist as discussed further.

A further consideration of laboratory testing is illustrated in FIG. 2. The first step is to define a “reference range” via statistical analysis of the population as is standard practice for laboratories. In FIG. 2 the reference range of serotonin is defined as 100 to 250 micrograms of serotonin per gram of creatinine. It is recognized that many people with urinary neurotransmitter assay values inside of the reference range are suffering from neurotransmitter dysfunction related illness and the only way to effective relief of symptoms is to establish neurotransmitter levels that are higher than the reference range in what is known as the therapeutic range. The Parkinson disease model illustrates very well why higher than normal levels are needed in many subjects not just in Parkinsonism. But still there is a subgroup of people who have no symptoms of neurotransmitter dysfunction and are functioning at a very high level. In studying this group of subjects, an “optimal range” was defined inside the reference range as illustrated in FIG. 2. Use of neurotransmitter assay in defining an optimal range for subjects who have no symptoms of neurotransmitter dysfunction is another application of this invention.

While the discussion so far has focused on urinary neurotransmitter assay applications the following considerations are made for salivary, serum, or other test methods that reflect the neurotransmitter status of the subject. With these other forms of assay there is also a flat dose-response curve as noted in FIG. 1 to the point of inflection at which time small increases in amino acid dosing leads to large increases in the neurotransmitters measured. With these other methods of assay there is also an optimal range that can be defined within the reference range.

All methods of assay when used properly insure that adequate amino acid therapy is given without over loading the system with neurotransmitters. With all methods the response to a given dose of amino acids varies widely. For example subjects have been seen who experience the inflection point of FIG. 1 on 50 milligrams per day of 5-HTP and others who do not experience the inflection point of FIG. 1 until 1,500 milligrams of 5-HTP per day is administered. When L-dopa and 5-HTP are used in combination for the synthesis of dopamine and serotonin respectively to affect proper balanced administration of amino acids, neurotransmitter levels of the system can be established that are well above the therapeutic range. When the other precursors of dopamine and serotonin are used, such as precursors of L-dopa and 5-HTP, in general neurotransmitters levels only into the therapeutic range can be established with a small probability of establishing neurotransmitter levels that are higher than the therapeutic range.

One aspect of the invention is to provide a neurotransmitter assay in support of amino acid therapy which insures that proper levels of neurotransmitters are established and when used properly minimizes the risks of neurotransmitter overload during use.

Another aspect of the invention provides a method of establishing at least one neurotransmitter status point in a subject comprising the steps of determining a subject'-s health status with respect to neurotransmitter dysfunction, performing an assay of a body fluid of the subject to determine a neurotransmitter level in the fluid, and defining the assayed neurotransmitter level in the fluid as at least one neurotransmitter status point.

Yet another aspect of the invention provides a method of treating a subject for neurotransmitter dysfunction, comprising the steps of performing a first assay of a body fluid of a subject to determine a baseline neurotransmitter level in the body fluid, administering an amino acid precursor of a neurotransmitter to the subject, administering a second assay of a body fluid of the subject to determine whether the neurotransmitter level in the body fluid is within a predetermined therapeutic range of neurotransmitter levels.

### BRIEF DESCRIPTION OF THE SEVERAL VIEWS OF THE DRAWING

FIG. 1 is a graph showing a relationship between urinary serotonin levels versus daily dosing of 5-HTP.

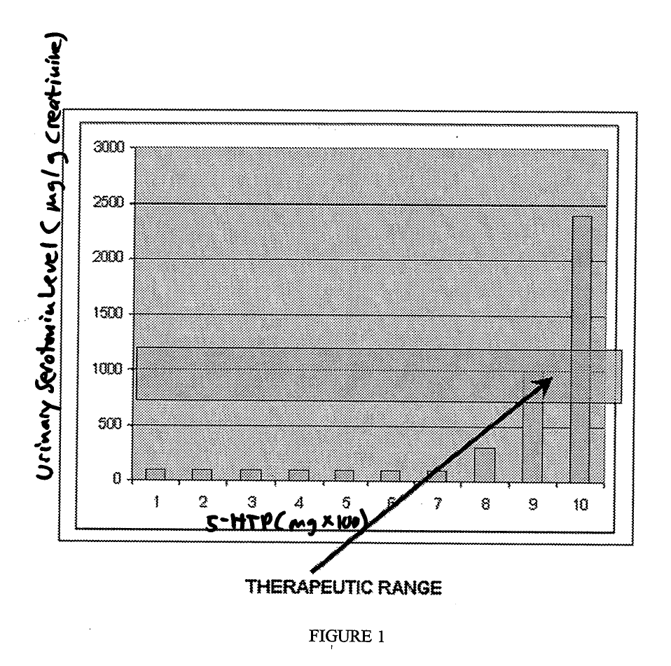

FIG. 2 illustrates a reference range of serotonin 100 to 250 micrograms of serotonin per gram of creatinine.

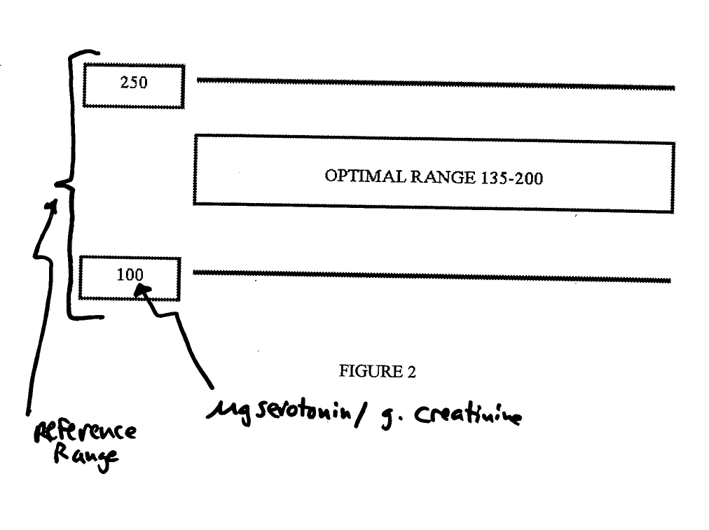

FIG. 3 is an illustration of the effects of using unopposed precursors of dopamine in treatment.

### DETAILED DESCRIPTION

The embodiment of the invention described is intended to be illustrative and not to be exhaustive or limit the invention to the exact forms disclosed. The embodiments are chosen and described so that persons skilled in the art will be able to understand the invention and the manner and process of making and using it.

The teachings of this invention, in general, relate to optimizing group outcomes in the treatment of the neurotransmitter system (The System) in the management of dysfunction in human beings as guided by laboratory assay of neurotransmitters. However, the teachings may also be useful in any life form where the catecholamine system and the serotonin system is found, such as other animals.

The catecholamine and serotonin systems as a whole are hereafter referred to as “The System”. The invention provides the ability to optimize group results in the treatment of The System related dysfunction via a safe and effective method to gain control of The System in the treatment of dysfunction, as well as to facilitate optimal function for systems dependant on the catecholamine and/or serotonin systems for regulation and function as guided laboratory assay of neurotransmitters of The System. Laboratory values and amino acid dosing listed in this description are for obtaining optimal results in a human population. Adjustment in dosing for non-human populations should be made based on body size and response as verified by laboratory assay.

### 1. General Discussion

A primary use of laboratory neurotransmitter assay is three fold:

1. To establish a “baseline” assay prior to treatment with amino acids.

2. To establish a “therapeutic level” of neurotransmitters in treatment with amino acids whereby the neurotransmitters are high enough in the system at the low point during the day to insure that symptoms of neurotransmitter dysfunction are not present and that neurotransmitter levels are not too high in the system so as to create other problems such as serotonin syndrome and the like.

3. To establish an “optimal level” of neurotransmitters in those subjects not suffering symptoms of neurotransmitter dysfunction.

In order to affect the three uses of neurotransmitters described immediately above, neurotransmitter assay via serum, saliva, urine, or other methods is used, as long as considerations of the limitations of each method as previously discussed are compensated for.

For saliva assay of neurotransmitters of The System, compensations are the need to perform several tests over a relatively short period of time (approximately 30 minutes) and averaging of the results.

For serum assay of neurotransmitters of The System, a compensation is the need to collect a sample where the subject is not disturbed so as to not affect the baseline neurotransmitter levels present just prior to collection of the sample.

For urinary assay of the neurotransmitters of The System, a compensation is a method of reporting results whereby the variability in the specific gravity of the urine is compensated for.

Methods for compensation in saliva, serum, and urine were previously discussed and the following is a discussion of interpretation and applications of neurotransmitter assay of neurotransmitters of The System.

The following laboratory value numbers are for the specific laboratory used in the research of this invention. Due to variability in assay techniques between laboratories actual values may legitimately vary from laboratory to laboratory.

“REFERENCE RANGES” are the ranges set by the individual laboratory from statistical analysis of a population of subjects based on defining the mean and standard deviation. An exemplary embodiment of the “reference range” is as follows:

Serotonin=100 to 250 micrograms of neurotransmitter per gram of creatinine.

Dopamine=100 to 250 micrograms of neurotransmitter per gram of creatinine.

Norepinephrine=25 to 75 micrograms of neurotransmitter per gram of creatinine.

Epinephrine=5 to 13 micrograms of neurotransmitter per gram of creatinine.

“OPTIMAL RANGES” are defined as a narrow range within the reference range where subjects with no symptoms of neurotransmitter dysfunction appear to be functioning optimally based on group observations. The “optimal ranges” for the neurotransmitters of The System are as follows:

Serotonin=175 to 225 micrograms of neurotransmitter per gram of creatinine.

Dopamine=125 to 175 micrograms of neurotransmitter per gram of creatinine.

Norepinephrine=30 to 55 micrograms of neurotransmitter per gram of creatinine.

Epinephrine=8 to 12 micrograms of neurotransmitter per gram of creatinine.

“THERAPEUTIC RANGES” are the range to be obtained in treatment to insure that resolution of symptoms is affected without overloading the system on neurotransmitters. The therapeutic ranges of the neurotransmitters of The System are as follows. It should be noted that these numbers are a relative guide only for reaching an inflection point in treatment and that the therapeutic range should not be fixed on the absolute numbers reported. For example, the therapeutic range for serotonin in non-obesity neurotransmitter disease is reported at 800 to 1,200. A serotonin level of 1,600 or higher could be acceptable in some circumstances.

Serotonin=1,200 to 2,400 micrograms of neurotransmitter per gram of creatinine for treatment of obesity.

Serotonin=250 to 1,200 micrograms of neurotransmitter per gram of creatinine for disease not related to obesity. Certain conditions such as panic attacks (panic disorder) and obsessive compulsive disorder may need higher levels established to affect resolution of symptoms.

Dopamine=200 to 600 micrograms of neurotransmitter per gram of creatinine.

Dopamine (in treatment of Parkinsonism) <20,000 micrograms of neurotransmitter per gram of creatinine and treatment is driven by clinical outcomes.

Norepinephrine=35 to 70 micrograms of neurotransmitter per gram of creatinine.

Epinephrine=8 to 13 micrograms of neurotransmitter per gram of creatinine.

The goal of treatment is to establish neurotransmitter levels of The System in the “optimal range” for subjects with no symptoms of neurotransmitter dysfunction and in the “therapeutic range” for subjects suffering from symptoms of neurotransmitter dysfunction. To affect establishment of neurotransmitters in the desired range, adjusting the dosing of amino acids is affected as described by applicant in US Patent Application Publication No. US 2003/0181509 A1, published Sep. 25, 2003.

### 2. Description of the Preferred Embodiment of the Invention

For neurotransmitter testing of neurotransmitters of The System, urine is collected approximately 5 to 6 hours prior to bed time and just prior to any amino acid dosing the subject may or may not be taking close to that time period. Once the urine sample is obtained, laboratory assay of the neurotransmitter of The System are performed as well as a urinary creatinine assay and the results are reported in terms of “micrograms of neurotransmitter per gram of creatinine”.

After there has been a start or change in the dosing of amino acid precursors of The System, the following considerations exist with regards to neurotransmitter assay.

1. It takes 3 to 5 days for serotonin levels to come to equilibrium in the urine.

2. It takes 3 to 5 days for dopamine levels to come to equilibrium in the urine.

3. It takes 2 to 6 weeks for norepinephrine to come to equilibrium in the urine.

4. It can take as long as 3 to 6 months for epinephrine to come to equilibrium in the urine.

If the subject is not under treatment with amino acid precursors of The System at the time the sample is collected, the results are used as a baseline reference point in treatment with amino acid precursors of The System.

If the subject has no symptoms of neurotransmitter dysfunction the subject is treated with amino acids precursors of The System and the amino acid dosing increased or decreased as guided by laboratory assay of neurotransmitters of The System until reported results of laboratory assay are in the optimal range.

If the subject is suffering from symptoms of neurotransmitter dysfunction, the subject is treated with amino acid precursors of The System which are in turn increased or decreased to until urinary neurotransmitter levels of The System have been established in the therapeutic range.

Retesting of the subject one week after every dose change in the amino acid precursors takes place should be done.

Once under treatment and optimal or therapeutic ranges have been established periodic retesting should be preformed at regular intervals. It is the preferred method to perform follow up testing every six months or sooner.

If assay of neurotransmitter reveals that the urinary neurotransmitter levels are low, the correct response is to increase the amino acid precursor dosing of The System. If assay of the neurotransmitters reveals that the urinary neurotransmitter levels are high the correct response is to decrease the amino acid precursor dosing of The System.

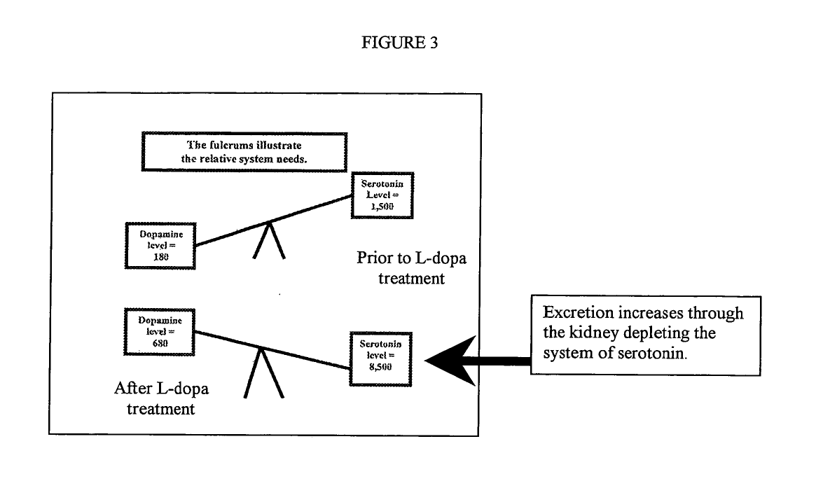

FIG. 3 illustrates the effects of using unopposed precursors of dopamine in treatment. With the addition of unopposed L-dopa the urinary excretion of serotonin increases markedly, a fact that was not previously known. The same is true with administration of unopposed precursors of the serotonin system such as 5-HTP. With the administration of 5-HTP alone there is a marked increase in the excretion of dopamine by the kidneys. These observations are of importance in establishing optimal and therapeutic ranges of neurotransmitters as guided by laboratory assay. For example, in a subject under treatment with amino acid precursors of The System who is experiencing symptoms of neurotransmitter dysfunction that are not obesity related and has a dopamine reported by assay of 150 micrograms of dopamine per gram of creatinine and a serotonin of 1,100 micrograms of serotonin per gram of creatinine and is still experience symptoms of neurotransmitter dysfunction, management considerations are follows.

1. By giving more of the “balanced” amino acid precursor of The System the urinary levels of both serotonin and dopamine levels will rise. This may lead to resolution of symptoms as the dopamine levels are established in the therapeutic range but the serotonin levels will be higher than the therapeutic or optimal range as well.

2. By administering only amino acid precursors of dopamine the ability is gained to establish dopamine levels in the therapeutic or optimal ranges. Referring to FIG. 3, by using unopposed amino acid precursors in treatment the excretion of serotonin by the kidneys increases markedly again leading to a situation where the dopamine is in the therapeutic range and the urinary serotonin levels are higher than the therapeutic or optimal ranges.

3. The proper management of a subject with 150 micrograms of dopamine per gram of creatinine and 1,100 micrograms of serotonin per gram of creatinine level is to increase the amino acid precursors of dopamine while at the same time decreasing the amino acid precursors of serotonin to give an outcome whereby neurotransmitters of both systems are in the therapeutic or optimal ranges.

Not all neurotransmitter dysfunction related symptoms resolve in the same therapeutic ranges. In general treatment of obesity, panic disorder, and obsessive compulsive disorder require urinary serotonin levels of 1,200 to 2,400 micrograms of serotonin per gram of creatinine. For diseases other than obesity, obsessive compulsive disorder, and panic disorder, serotonin levels of 250 to 1,200 micrograms of serotonin per gram of creatinine are the usual range. In the treatment of Parkinsonism the therapeutic range for serotonin is 250 to 1,200 micrograms per gram of creatinine and the therapeutic range is to elevate the dopamine levels high enough to get the symptoms of Parkinsonism under control. In general in treatment of Parkinsonism the therapeutic range is to keep the dopamine levels less than 20,000 micrograms of dopamine per gram of creatinine. Although preferred numbers for a therapeutic range are provided here, it should be understood that variance in lab techniques may change these numbers and that testing should be used as a guide to insure that the inflection point has been reached without overloading the system. It should be understood that recognition of an inflection point during treatment is an important consideration.

Simply establishing urinary neurotransmitter levels in the therapeutic range may not control symptoms in all subjects. For example, in treatment of the obese subject with a urinary serotonin assay of 2,300 micrograms of serotonin per gram of creatinine and a dopamine level of 475 micrograms of dopamine per gram of creatinine with the norepinephrine and epinephrine levels in the therapeutic ranges as well and the subject is not losing weight considerations are a follows. In a case such as this from a neurotransmitter standpoint of The System further treatment may be limited and other causes that are preventing the subject from losing weight should be considered. In most cases such as this there is a major stressor in the subjects life that can be identified which is distracting them from doing the things they need to do to be successful at weight loss. Considerations such as this also apply to other neurotransmitter dysfunction symptoms of illness.

1. A method of establishing at least one neurotransmitter status point in a subject, comprising the steps of determining a subject'-s health status with respect to neurotransmitter dysfunction, performing an assay of a body fluid of the subject to determine a neurotransmitter level in the fluid, and defining the assayed neurotransmitter level in the fluid as at least one neurotransmitter status point.

2. The method of claim 1, wherein the subject is a human being.

3. The method of claim 1, wherein the step of determining the subject's health status is implemented by a medical examination.

4. The method of claim 1, wherein health status is determined with respect to the group of dysfunction consisting of obesity, panic disorder, obsessive compulsive disorder, and Parkinson's disease.

5. The method of claim 1, wherein the step of assaying is implemented via the subject's serum fluid.

6. The method of claim 1, wherein the step of assaying is implemented via the subject's saliva fluid.

7. The method of claim 1, wherein the step of assaying is implemented via the subject's urine fluid.

8. The method of claim 7, wherein the urine for assay is collected from the subject approximately 5-6 hours before the subject's bedtime.

9. The method of claim 7, wherein the step of assaying measures neurotransmitter in micrograms of neurotransmitter per gram of creatinine in urine.

10. The method of claim 1, wherein the neurotransmitter is serotonin.

11. The method of claim 1, wherein the neurotransmitter is catecholamine.

12. The method of claim 1, wherein the neurotransmitter is serotonin and catecholamine.

13. The method of claim 1, wherein the at least one neurotransmitter status point is a baseline reference point.

14. The method of claim 13, wherein the baseline reference point is within a reference range.

15. The method of claim 14, wherein the reference range for concentrations of serotonin neurotransmitter is approximately 100-250 micrograms of neurotransmitter per gram of creatinine.

16. The method of claim 14, wherein the reference range of dopamine amino acid precursor of catecholamine neurotransmitter is approximately 100-250 micrograms of neurotransmitter per gram of creatinine.

17. The method of claim 14, wherein the reference range of norepinephrine amino acid precursor of catecholamine neurotransmitter is approximately 25-75 micrograms of neurotransmitter per gram of creatinine.

18. The method of claim 14, wherein the reference range of epinephrine amino acid precursor of catecholamine neurotransmitter is approximately 5-13 micrograms of neurotransmitter per gram of creatinine.

19. The method of claim 14, wherein the baseline reference point is further within an optimal range.

20. The method of claim 19, wherein the optimal range for concentrations of serotonin neurotransmitter is approximately 175-225 micrograms of neurotransmitter per gram of creatinine.

21. The method of claim 19, wherein the optimal range of dopamine amino acid precursor of catecholamine neurotransmitter is approximately 125-175 micrograms of neurotransmitter per gram of creatinine.

22. The method of claim 19, wherein the optimal range of norepinephrine amino acid precursor of catecholamine neurotransmitter is approximately 30-55 micrograms of neurotransmitter per gram of creatinine.

23. The method of claim 19, wherein the optimal range of epinephrine amino acid precursor of catecholamine neurotransmitter is approximately 8-12 micrograms of neurotransmitter per gram of creatinine.

24. The method of claim 13, wherein the baseline reference point is outside a reference range.

25. The method of claim 1, wherein the at least one neurotransmitter status point is a therapeutic point.

26. The method of claim 25, wherein the at least one therapeutic point is within a therapeutic range of concentrations of neurotransmitter.

27. The method of claim 26, wherein the therapeutic range for concentrations of serotonin neurotransmitter is approximately 1,200-2,400 micrograms of neurotransmitter per gram of creatinine, for treatment of obesity.

28. The method of claim 26, wherein the therapeutic range for concentrations of serotonin neurotransmitter is approximately 250-1,200 micrograms of neurotransmitter per gram of creatinine, for treatment related to panic disorder and obsessive compulsive disorder.

29. The method of claim 26, wherein the therapeutic range of dopamine amino acid precursor of catecholamine neurotransmitter is approximately 200-500 micrograms of neurotransmitter per gram of creatinine.

30. The method of claim 26, wherein the therapeutic range of dopamine amino acid precursor of catecholamine neurotransmitter is approximately <20,000 micrograms of neurotransmitter per gram of creatinine for treatment of Parkinsonism.

31. The method of claim 26, wherein the therapeutic range of norepinephrine amino acid precursor of catecholamine neurotransmitter is approximately 35-70 micrograms of neurotransmitter per gram of creatinine.

32. The method of claim 26, wherein the therapeutic range of epinephrine amino acid precursor of catecholamine neurotransmitter is approximately 8-13 micrograms of neurotransmitter per gram of creatinine.

33. The method of claim 25, further comprising the step of treating the subject after the assay step, and wherein the administration step is repeated with increasing amounts of amino acid precursors, each administration step being followed by an assay step, and further comprising the step of graphing neurotransmitter level over time.

34. The method of claim 33, further comprising the step of determining an inflection point on the graph of neurotransmitter level.

35. The method of claim 34, wherein the inflection point is used to determine the therapeutic range.

36. A method of establishing at least one neurotransmitter status point in a human being, comprising the steps of:
a. determining a subject'-s health status with respect to catecholamine-serotonin system neurotransmitter dysfunction by medical examination for symptoms of dysfunction;
b. performing an assay of a body fluid of the subject to determine a catecholamine-serotonin system neurotransmitter level in the fluid, the assay being a urinary assay and the urine sample being collected from the subject about 5-6 hours before the subject's bedtime; and
c. defining the assayed catecholamine-serotonin system neurotransmitter level in the fluid as at least one neurotransmitter status point.

37. A method of treating a subject for neurotransmitter dysfunction, comprising the steps of performing a first assay of a body fluid of a subject to determine a baseline neurotransmitter level in the body fluid, administering an amino acid precursor of a neurotransmitter to the subject, administering a second assay of a body fluid of the subject to determine whether the neurotransmitter level in the body fluid is within a predetermined therapeutic range of neurotransmitter levels.

38. The method of claim 37, wherein the subject is a human being, and wherein health status is determined with respect to the group of dysfunctions consisting of obesity, panic disorder, obsessive compulsive disorder, and Parkinson's disease.

39. The method of claim 38, wherein the step of assaying is implemented via the subject's serum fluid.

40. The method of claim 37, wherein the step of assaying is implemented via the subject's saliva fluid.

41. The method of claim 37, wherein the step of assaying is implemented via the subject's urine fluid.

42. The method of claim 41, wherein the urine for assay is collected from the subject approximately 5-6 hours before the subject's bedtime.

43. The method of claim 41, wherein the step of assaying measures neurotransmitter in micrograms of neurotransmitter per gram of creatinine in urine.

44. The method of claim 43, wherein the neurotransmitter is selected from the group of neurotransmitters consisting of serotonin, catecholamine, and a combination of serotonin and catecholamine.

45. The method of claim 44, wherein the therapeutic range for concentrations of serotonin neurotransmitter is approximately 1,200-2,400 micrograms of neurotransmitter per gram of creatinine, for treatment of obesity.

46. The method of claim 44, wherein the therapeutic range for concentrations of serotonin neurotransmitter is approximately 250-1,200 micrograms of neurotransmitter per gram of creatinine, for treatment related to panic disorder and obsessive compulsive disorder.

47. The method of claim 44, wherein the therapeutic range of dopamine amino acid precursor of catecholamine neurotransmitter is approximately 200-500 micrograms of neurotransmitter per gram of creatinine.

48. The method of claim 44, wherein the therapeutic range of dopamine amino acid precursor of catecholamine neurotransmitter is approximately <20,000 micrograms of neurotransmitter per gram of creatinine for treatment of Parkinsonism.

49. The method of claim 44, wherein the therapeutic range of norepinephrine amino acid precursor of catecholamine neurotransmitter is approximately 35-70 micrograms of neurotransmitter per gram of creatinine.

50. The method of claim 44, wherein the therapeutic range of epinephrine amino acid precursor of catecholamine neurotransmitter is approximately 8-13 micrograms of neurotransmitter per gram of creatinine.

51. The method of claim 43, wherein the administration step is repeated with increasing amounts of amino acid precursors, each administration step being followed by an assay step, and further comprising the step of graphing neurotransmitter level over time to determine an inflection point on the graph of neurotransmitter level, and wherein the inflection point is used to determine the therapeutic range.

52. The method of claim 51, further comprising the step of increasing or decreasing the amount of amino acid precursor in the administration step to maintain the level of neurotransmitter in the therapeutic range.

53. A method of treating a human being for obesity, panic disorder, obsessive-compulsive disorder, Parkinson's disease or the like based on catecholamine-serotonin neurotransmitter dysfunction, comprising the steps of:
a. performing a first assay of a body fluid of a patient to determine a baseline neurotransmitter level in the body fluid, the assay being a urinary assay and the urine sample being collected from the patient about 5-6 hours before the patient's bedtime;
b. administering an amino acid precursor of a neurotransmitter to the subject;
c. administering a second assay of a body fluid of the subject to determine whether the neurotransmitter level in the body fluid is within a predetermined therapeutic range of neurotransmitter levels; wherein the administration step is repeated with increasing amounts of amino acid precursors, each administration step being followed by an assay step; and
d. graphing neurotransmitter level over time to determine an inflection point on the graph of neurotransmitter level, and wherein the inflection point is used to determine the therapeutic range.
